(19) United States
(12) Patent Application Publication
(10) Pub. No.: US 2014/0180220 A1
(43) Pub. Date: Jun. 26, 2014

(54) Tunable Nonfouling Surface Of Oligoethylene Glycol

(71) Applicant: Duke University, Durham, NC (US)

(72) Inventors: Ashutosh Chilkoti, Durham, NC (US); Hongwei Ma, Hangzhou (CH)

(21) Appl. No.: 13/943,351

(22) Filed: Jul. 16, 2013

Related U.S. Application Data
(63) Continuation of application No. 10/783,054, filed on Feb. 20, 2004, now abandoned.

(51) Int. Cl. A61L 29/08; A61L 31/14; A61L 29/14; A61L 31/10

(52) U.S. Cl. CPC A61L 29/085; A61L 31/10; A61L 31/14; A61L 29/14
USPC 604/265; 428/319.3; 428/36.91

(57) ABSTRACT

An article having a nonfouling surface thereon is comprises: (a) a substrate having a surface portion; (b) a linking layer on the surface portion; and (c) a polymer layer formed on the linking layer, preferably by the process of surface-initiated polymerization of monomeric units thereon, with each of the monomeric units comprising a monomer core group having at least one protein-resistant head group coupled thereto, to thereby form a brush molecule on the surface portion. The brush molecule comprising a stem formed from the polymerization of the monomer core groups, and a plurality of branches formed from the hydrophilic head group projecting from the stem. Methods of making and using such articles, are also described.

### STATEMENT OF GOVERNMENT SUPPORT

This invention was made with government support under Grant No. EEC-0210590 from the National Science Foundation and Grant No. DBI-0098534 from the National Science Foundation. The United States Government has certain rights to this invention.

### FIELD OF THE INVENTION

The present invention concerns articles having a nonfouling surface coating thereon, methods of making the same, and methods of using the same. The invention may be utilized with a variety of different types of articles that contact a fluid, particularly a biological fluid such as blood, that would otherwise be subject to fouling.

### BACKGROUND OF THE INVENTION

The ability of surface coatings containing poly(ethylene glycol) (PEG) to prevent nonspecific protein adsorption and cell adhesion have been recognized for decades and have resulted in many biomedical applications of this class of materials (Harris, J. M. in Poly(Ethylene Glycol) Chemistry: Biotechnical and BiomedicalApplications (Ed: Harris, J. M.), Plenum Press, New York, 1992, 1-14).

Self-assembled monolayers of oligo(ethylene glycol)-terminated alkanethiols [(EG)ₙ-SH SAM)] present a dense “nonfouling” brush that confers protein resistance to gold, and are arguably the best nonfouling systems that are currently available. Unfortunately these systems are characterized by limited robustness (Mrksich, M., Dike, L. E., Tien, J., Ingber, D. E., Whitesides, G. M., Exp. Cell Res. 1997, 235, 305; Mrksich, M., Whitesides, G. M. in American Chemical Society Symposium Series on Chemistry and Biological Applications of Polyethylene Glycol 680 (Eds: Harris, J. M. & Zalipsky, S.), Washington D.C., ACS, 1997, 361-373, and references therein).

Common methods to immobilize PEG include physisorption (Lee, J. H., Andrade, J. D. in Polymer Surface Dynamics (Ed: Andrade, J. D.), Plenum Press, New York, 1988, 119-136; Lee, J. H., Kopecek, J., Andrade, J. D., J. Biomed. Mater. Res. 1989, 23, 351; Elbert, D. L., Hubbell, J. A., J. Biomed. Mater. Res. 1998, 42, 55; Liu, V. A., Jascromb, W. E., Bhatia, S, N., J. Biomed. Mater. Res. 2002, 60, 126), chemisorption (Prime, K. L., Whitesides, G. M., J. Am. Chem. Soc. 1993, 15, 10714; Xia, N., Hu, Y. H., Grainger, D. W., Castner, D. G., Langmuir 2002, 8, 3255; Bearinger, J. P. et al., Nat. Mater. 2003, 2, 259), and covalent grafting (Nojiri, C. et al., J. Biomed. Mater. Res. 1990, 24, 1151; Sun, Y. H., Gombotz, W. R., Hoffman, A. S., J. Bioactive Compat. Polym. 1986, 1, 316; Merrill, E. W. et al. in Polymers In Medicine: Biomedical & Pharmaceutical Applications (Eds: Ottenbrite, R. M., Chiellini, E.), Technomic Lancaster, Pa., 1992, 39-56) of PEG onto surfaces; more exotic methods include plasma polymerization of oligoethylene glycol precursors (López, G. P. et al., J. Biomed. Mater. Res. 1992, 26, 415). Physisorption or covalent grafting (the “grafting to” approach) results in a low surface density of PEG chains, which limits their protein and cell resistance. In contrast, although (EG)ₙ-SH SAMs on gold exhibit significantly better protein and cell resistance than grafted PEG, they have several limitations; because SAMs are a single molecular layer, they have limited robustness, which is further exacerbated by the existence of defects in the SAM (Kim, Y. T., Bard, A. J., Langmuir 1992, 8, 1096; Schönenberger, C., Sondag-huethorst, J. A. M., Jorritsma, J., Fokkink, L. G., Langmuir 1994, 10, 611; Zhao, X. M., Wilbur, J. L., Whitesides, G. M., Langmuir 1996, 12, 3257) and the propensity of the chemisorbed thiolate to oxidize (Tarlov, M. J., Newman, J. G., Langmuir 1992, 8, 1398; Tarlov, M. J., Newman, J. G., Langmuir 1992, 8, 1398). These factors contribute to the loss of cell resistance after a week in culture (Mrksich, M., Dike, L. E., Tien, J., Ingber, D. E., Whitesides, G. M., Exp. Cell Res. 1997, 235, 305).

Accordingly, there is a need for new ways to provide a nonfouling surface coating on articles.

### SUMMARY OF THE INVENTION

A first aspect of the present invention is an article having a nonfouling surface thereon, the article comprising:

(a) a substrate having a surface portion;

(b) a linking layer on the surface portion; and

(c) a polymer layer formed on the linking layer, preferably by the process of surface-initiated polymerization of monomeric units thereon, with each of the monomeric units comprising a monomer (for example, a vinyl monomer) core group having at least one protein-resistant head group coupled thereto, to thereby form a brush molecule on the surface portion. The brush molecule comprising a stem formed from the polymerization of the monomer core groups, and a plurality of branches formed from the hydrophilic head group projecting from the stem.

A second aspect of the present invention is a method of making an article having a nonfouling surface thereon, the method comprising: (a) providing a substrate having a surface portion; (b) depositing a linking layer on the surface portion; and (c) forming a polymer layer on the linking layer by the process of surface-initiated polymerization of monomeric units thereon, with each of the monomeric units comprising a monomer (for example, a vinyl monomer) core group having at least one protein-resistant head group coupled thereto, to thereby form a brush molecule on the surface portion; the brush molecule comprising a stem formed from the polymerization of the monomer core groups, and a plurality of branches formed from the hydrophilic head group projecting from the stem.

In some embodiments of the invention, the surface portion comprises a material selected from the group consisting of metals, metal oxides, semiconductors, polymers, silicon, silicon oxide, and composites thereof.

In some embodiments of the invention the linking layer is continuous; in some embodiments of the invention the linking layer is patterned. In some embodiments of the invention the linking layer is a self-assembled monolayer. In some embodiments of the invention the linking layer comprises an initiator-terminated alkanethiol.

In some embodiments of the invention the surface-initiated polymerization is carried out by atom transfer radical polymerization; in some embodiments of the invention the surface-initiated polymerization is carried out by free radical polymerization.

In some embodiments, the article further comprises a protein, peptide, oligonucleotide or peptide nucleic acid covalently coupled to the brush molecule. In some embodiments the protein, peptide, oligonucleotide or peptide nucleic acid coupled to the brush molecule or to the surface consist of or consist essentially of a single preselected molecule (this is, one such molecule is coupled to the surface portion via the brush molecule, to the exclusion of other different molecules). The preselected molecule may be a member of a specific binding pair, such as a receptor.

Still other aspects of the present invention are explained in greater detail below.

### BRIEF DESCRIPTION OF THE DRAWINGS

FIG. 1. Surface-initiated polymerization. (A) Molecular structure of initiator (1), diluent thiol (2), monomer (OEGMA), and a tethered “bottle” brush of poly(OEGMA) grown from a mixed SAM of (1) and (2). (B) Ellipsometric thickness of the poly(OEGMA) brush as a function of polymerization time. Polymer brushes were grown from the surface of a pure SAM of (1), and exhibit linear growth kinetics for a polymerization time of upto 120 min. The sd for each data point is <3 Å (n=3). (C) Poly(OEGMA) brushes were grown from mixed SAMs of (1) and (2) for a polymerization time of 40 min, and a saturation point in thickness was observed at a bulk mole fraction of (1) of 0.6 (χ1); sd for each data point is <4 Å.

FIG. 2. Surface plasmon resonance (SPR). SPR chips were coated with a poly(OEGMA) brush grown from a pure SAM of (1) for a polymerization time of 40 min: (A) after priming with PBS buffer for 10 min (region I), 10% FBS (red curve), 1 mg ml-1 fibronectin (blue curve), or 100% FBS solution (green curve) were injected over the surface (at 10 min: indicated by II) for 20 min (region III), followed by a 10 min rinse with PBS (region IV).

FIG. 3. Patterns of poly(OEGMA) brush and attached cells. (A) SEM image of a patterned poly(OEGMA) brush on gold that was fabricated by μCP of (1) followed by SIATRP (160 min) of OEGMA. (B) 3-dimensional image of a poly(OEGMA) nanoarray over a 5×5 um² area grown from the initiator thiol (1) patterned with DPN on gold. (C) The line profile of (B) shows that the poly(OEGMA) nanostructures have a diameter of ˜90 nm and a height of ˜14 nm. (D) and (E) NIH 3T3 cells seeded onto a pattern of adsorbed fibronectin (20 um circles (D) and 40 um stripes (E)) separated by cell-resistant regions of poly(OEGMA) brushes fabricated by SI-ATRP on gold (40 um (D) and (E)).

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

### 1. DEFINITIONS

“Biological fluid” as used herein may be any fluid of human or animal origin, including but not limited to blood, blood plasma, peritoneal fluid, cerebrospinal fluid, tear, mucus, and lymph fluid. Biological fluids generally contain a mixture of different proteins therein, and typically contain other constituents such as other cells and molecules. Biological fluids may be in their natural state or in a modified state by the addition of ingredients such as reagents or removal of one or more natural constituents (e.g., blood plasma).

“Kosmotrope”, while originally used to denote a solute that stabilized a protein or membrane, is also used (and is used herein) to denote a substituent or “head group” which, when deposited on a surface, renders that surface protein-resistant. See, e.g., R. Kane. P. Deschatelets and G. Whitesides, Kosmotropes Form the Basis of Protein-Resistant Surfaces, Langmuir 19, 2388-2391 (2003).

“Polymer” as used herein is intended to encompass any type of polymer, including homopolymers, heteropolymers, co-polymers, ter-polymers, etc., and blends, combinations and mixtures thereof.

“Specific binding pair” as used herein refers to two compounds that specifically bind to one another, such as (functionally): a receptor and a ligand (such as a drug), an antibody and an antigen, etc.; or (structurally): protein or peptide and protein or peptide; protein or peptide and nucleic acid; etc.

### 2. SUBSTRATES

The present invention can be utilized to form non-fouling surfaces on a variety of different types of substrates. Preferably the substrates are ones in which the article, particularly the coated surface portion, contacts a biological fluid, either in vivo or ex vivo.

In one embodiment, the article is a contact lens or intra-ocular lens, and the surface portion is a surface portion thereof that would be in contact with a body fluid. Examples of such articles include but are not limited to those described in U.S. Pat. Nos. 6,659,607; 6,649,722; 6,634,753; 6,627,674; RE38,193; 6,692,525; 6,666,887; 6,645,246; 6,645,245; and 6,638,305.

In another embodiment, the article is an orthopedic implant such as a replacement joint (e.g., finger, knee, hip), disc, vertebra, pin, screw, rod, etc. Examples of such articles include but are not limited to those described in U.S. Pat. Nos. 6,602,293; 6,520,996; 6,621,291; 5,973,222; 5,906,644; 5,507,814; 5,443,513; and 5,092,893.

In another embodiment, the article is a vascular graft (e.g., a synthetic vascular graft) or a stent. Examples of such articles include but are not limited to those described in U.S. Pat. Nos. 6,491,718; 6,471,721; 6,293,968; 6,187,035; 6,165,209; 6,652,570; 6,605,113; 6,517,571; 6,436,135; 6,428,571; 6,120,532; and 5,747,128.

In another embodiment, the article is a shunt or catheter (e.g., a chronic or indwelling shunt or catheter). Examples of such articles include but are not limited to those described in U.S. Pat. Nos. 6,544,208; 5,683,434; 4,867,740; 4,861,331; 6,471,689; 5,809,354; 5,800,498; 5,755,764; 5,713,858; and 5,688,237.

In another embodiment, the article is a dialysis machine or blood oxygenator (including component parts thereof). In this case, the surface is a blood contact or other biological fluid contact surface. Examples of such articles include but are not limited to those described in U.S. Pat. Nos. 6,623,442; 6,620,118; 6,595,948; 6,595,948; 6,447,488; 6,290,669; 6,284,131; 6,602,467; 6,576,191; 6,454,999; 6,387,324; 6,350411; and 6,224,829.

In still other embodiments, the article is an implantable electrical lead, an implantable electrode, an implantable pacemaker, or an implantable cardioverter (e.g., an implantable defibrillator). Examples of such articles include but are not limited to those described in U.S. Pat. Nos. 6,671,553; 6,650,945; 6,640,136; 6,636,770; 6,633,780; 6,606,521; 6,580,949; 6,574,505; 6,493,591; 6,477,427; and 6,456,876.

In still other embodiments, the article is a label-free optical or mass detector (e.g., a surface plasmon resonance energy detector, an optical wave guide, an ellipsometry detector, etc.) and the surface is a sensing surface (e.g., a surface portion that would be in contact with a biological fluid). Examples of such articles include but are not limited to those described in U.S. Pat. Nos. 6,579,721; 6,573,107; 6,570,657; 6,423,055; 5,991,048; 5,822,073; 5,815,278; 5,625,455; 5,485,277; 5,415,842; 4,844,613; and 4,822,135.

In still other embodiments, the article is a biosensor, an assay plate, or the like. For example, the present invention may be utilized with optical biosensors such as described in U.S. Pat. No. 5,313,264 to Ulf et al., U.S. Pat. No. 5,846,842 to Herron et al., U.S. Pat. No. 5,496,701 to Pollard-Knight et al., etc. The present invention may be utilized with potentiometric or electrochemical biosensors, such as described in U.S. Pat. No. 5,413,690 to Kost, or PCT Application WO98/35232 to Fowlkes and Thorp. The present invention may be utilized with a diamond film biosensor, such as described in U.S. Pat. No. 5,777,372 to Kobashi. Thus, the solid support may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. For example, the solid support may be comprised of polyethylene, polytetrafluoroethylene, gold, silicon, silicon oxide, silicon oxynitride, indium, platinum, iridium, indium tin oxide, diamond or diamond-like film, etc.

The present invention may be utilized with substrates for “chip-based” and “pin-based” combinatorial chemistry techniques. All can be prepared in accordance with known techniques. See e.g., U.S. Pat. No. 5,445,934 to Fodor et al., U.S. Pat. No. 5,288,514 to Ellman, and U.S. Pat. No. 5,624,711 to Sundberg et al., the disclosures of which are incorporated by reference herein in their entirety.

Substrates as described above can be formed of any suitable material, including but not limited to comprises a material selected from the group consisting of metals, metal oxides, semiconductors, polymers (particularly organic polymers in any suitable form including woven, nonwoven, molded, extruded, cast, etc.), silicon, silicon oxide, and composites thereof.

Polymers used to form substrates as described herein may be any suitable polymer, including but not limited to: poly(ethylene) (PE), poly(propylene) (PP), cis and trans isomers of poly(butadiene) (PB), cis and trans isomers of poly(ispoprene), poly(ethylene terephthalate) (PET), polystyrene (PS), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), fluorinated polyacrylate (PFOA), poly(ethylene-butylene) (PEB), poly(styrene-acrylonitrile) (SAN), polytetrafluoroethylene (PTFE) and its derivatives, polyolefin plastomers, and combinations and copolymers thereof, etc.

If desired or necessary, the substrate may have an additional layer such as a gold or an oxide layer formed on the relevant surface portion to facilitate the deposition of the linking layer, as discussed further below.

### 3. LINKING (OR “ANCHOR”) LAYERS

Anchor layers used to carry out the present invention are generally formed from a compound comprising an anchor group coupled (e.g., covalently coupled) to an initiator (e.g., directly coupled or coupled through an intermediate linking group). The choice of anchor group will depend upon the surface portion on which the linking layer is formed, and the choice of initiator will depend upon the particular reaction used to form the brush polymer as discussed in greater detail below.

The anchoring group may be selected to covalently or non-covalently couple the compound or linking layer to the surface portion. Non-covalent coupling may be by any suitable secondary interaction, including but not limited to hydrophobic bonding, hydrogen bonding, Van der Waals interactions, ionic bonding, etc. Examples of substrate materials and corresponding anchoring groups include, for example, gold, silver, copper, cadmium, zinc, palladium, platinum, mercury, lead, iron, chromium, manganese, tungsten, and any alloys thereof with sulfur-containing functional groups such as thiols, sulfides, disulfides (e.g., —SR or —SSR where R is H of alkyl, typically lower alkyl, or aryl), and the like; doped or undoped silicon with silanes and chlorosilanes (e.g., —SiR₂Cl wherein R is H or alkyl, typically lower alkyl, or aryl); metal oxides such as silica, alumina, quartz, glass, and the like with carboxylic acids as anchoring groups; platinum and palladium with nitrites and isonitriles; and copper with hydroxamic acids. Additional suitable functional groups suitable as the anchoring group include benzophenones, acid chlorides, anhydrides, epoxides, sulfonyl groups, phosphoryl groups, hydroxyl groups, amino acid groups, amides, and the like. See, e.g., U.S. Pat. No. 6,413,587.

Any suitable initiator may be incorporated into the anchoring group by introduction of a covalent bond at a location non-critical for the activity of the initiator. Examples of such initiators include, but are not limited to, bromoisobutyrate, polymethyl methacrylate-Cl, polystyrene-Cl, AIBN, 2-bromoisobutyrate, chlorobenzene, hexabromomethyl benzene, hexachloromethyl benzene, dibromoxylene, methyl bromopropionate. Additional examples of initiators include those initators described in U.S. Pat. No. 6,413,587 to Hawker (particularly at columns 10-11 thereof) and those initiators described in U.S. Pat. No. 6,541,580 to Matyjaszewski et al.

As noted above, a linking group or “spacer” may be inserted between the anchoring group and initiator. The linker may be polar, nonpolar, positively charged, negatively charged or uncharged, and may be, for example, saturated or unsaturated, linear or branched alkylene, aralkylene, alkarylene, or other hydrocarbylene, such as halogenated hydrocarbylene, particularly fluorinated hydrocarbylene. Preferred linkers are simply saturated alkylene of 3 to 20 carbon atoms, i.e., —(CH₂)₄— where n is an integer of 3 to 20 inclusive. See, e.g., U.S. Pat. No. 6,413,587. Another preferred embodiment of the linkes is an oligoethyleneglycol of 3 to 20 units, i.e., (CH₂CH₂O)ₙ where n ranges from 3 to 20.

The anchoring layer may be deposited by any suitable technique. It may be deposited as a self-assembled monolayer. It may be created by modification of the substrate by chemical reaction (see, e.g., U.S. Pat. No. 6,444,254 to Chilkoti et al.) or by reactive plasma etching or corona discharge treatment. It may be deposited by a plasma deposition process. It may be deposited by deposition, printing, stamping, etc. It may be deposited as a continuous layer or as a discontinuous (e.g., patterned) layer.

### 4. BRUSH POLYMER FORMATION

The brush polymers are, in general, formed by the polymerization of monomeric core groups having a protein-resistant head group coupled thereto.

Any suitable core vinyl monomer polymerizable by the processes discussed below can be used, including but not limited to styrenes, acrylonitriles, acetates, acrylates, methacrylates, acrylamides, methacrylamides, vinyl alcohols, vinyl acids, and combinations thereof.

Protein resistant groups may be hydrophilic head groups or kosmotropes. Examples include but are not limited to oligosaccharides, tri(propyl sulfoxide), phosphorylcholine, tri(sarcosine) (Sarc), N-acetylpiperazine, permethylated sorbitol, hexamethylphosphoramide, an intramolecular zwitterion (for example, —CH₂N⁺(CH₃)₂CH₂CH₂CH₂SO₃⁻) (ZW), and mannitol.

Additional examples of kosmotrope protein resistant head groups include, but are not limited to:

-(EG)₆OH;

—O(Mannitol);

—C(O)N(CH₃)CH₂(CH(OCH₃))₄CH₂OCH₃;

—N(CH₃)₃⁺Cl⁻/—SO₃⁻Na⁺;

—N(CH₃)₂⁺CH₂CH₂SO₃;

—C(O)Pip(NAc);

—N(CH₃)₂⁺CH₂CO₂⁻;

—O([Blc-α(1,4)-Glc-β(1)-]);

—C(O)(N(CH₃)CH₂C(O))₃N(CH₃)₂;

—N(CH₃)₂⁺CH₂CH₂CH₂SO₃⁻;

—C(O)N(CH₃)CH₂CH₂N(CH₃)P(O)(N(CH₃)₂)₂; and

—(S(O)CH₂CH₂CH₂)₃S(O)CH₃.

### See, e.g., R. Kane et al., Langmuir 19, 2388-91 (2003)(Table 1).

A particularly preferred protein resistant head group is poly(ethylene glycol), or “PEG”, for example PEG consisting of from 3 to 20 monomeric units.

Free radical polymerization of monomers to form brush polymers can be carried out in accordance with known techniques, such as described in U.S. Pat. No. 6,423,465 to Hawker et al.; U.S. Pat. No. 6,413,587 to Hawker et al.; U.S. Pat. No. 6,649,138 to Adams et al.; US Patent Application 2003/0108879 to Klaemer et al.; or variations thereof which will be apparent to skilled persons based on the disclosure provided herein

Atom or transfer radical polymerization of monomers to form brush polymers can be carried out in accordance with known techniques, such as described in U.S. Pat. No. 6,541,580 to Jatyjaszewski et al.; U.S. Pat. No. 6,512,060 to Matyjaszewski et al.; or US Patent Application 2003/0185741 to Jatyjaszewski et al., or variations thereof which will be apparent to skilled persons based on the disclosure provided herein.

In general, the brush molecules formed by the processes described herein will be from 2 or 5 up to 50 or 100 nanometers in length, or more, and will be deposited on the surface portion at a denisty of from 10, 20 or 40 up to 100, 200 or 500 milligrams per meter², or more.

### 5. USES AND APPLICATIONS OF ARTICLES

A further aspect of the present invention is a method of using an article as described herein, comprising: (a) providing an article as described above; and then (b) contacting the article to a biological fluid, and where proteins in the fluid do not bind to the surface portion. The contacting step may be carried out in vivo (e.g., by implanting an orthopedic implant, lead, catheter, shunt, stent, vascular graft intraocular lens or the like into a human or animal subject, or inserting a contact lens onto the eye of a human or animal subject) or may be carried out ex vivo (e.g., by passing a biological fluid such as blood through a dialysis apparatus or blood oxygenator, by passing a biological fluid into a detector). The contacting step may be carried out acutely or chronically: e.g., for a period of at least one day, one week, one month, one year, etc., depending upon the particular article being utilized.

In some embodiments the present invention is utilized by (a) providing an article as described herein, the article further comprising a first member of a specific binding pair such as a protein, peptide, oligonucleotide, peptide nucleic acid or the like covalently coupled to the brush molecule, the first member preferably consisting essentially of a single preselected molecule; and then (b) contacting the article to a biological fluid, the biological fluid containing a second member of the specific binding pair, wherein the second member of the specific binding pair binds to the surface portions, and where other proteins or peptides in the fluid do not bind to the surface portion. Such uses are particularly appropriate where the article is a sensor or biosensor as described in greater detail above.

The present invention is explained in greater detail in the following non-limiting Examples.

### EXAMPLES

The present invention provides, among other things, methods to synthesize nonfouling coatings that in some embodiments combine the advantages of SAMs, namely their high surface density and ease of formation, with those of polymers—thicker, more robust films and versatile architecture and chemistry—are of significant interest for a variety of applications. Among other things, we show herein that (EG)ₙ functionalized polymer brushes of tunable thickness in the 5-50 nm range, a thickness inaccessible to SAMs or polymer grafts, can be easily synthesized by surface-initiated polymerization (SIP) (Zhao, B., Brittain, W. J., Prog. Polym. Sci. 2000, 25, 677, and references therein), that these polymer brushes exhibit no detectable adsorption of proteins and are cell-resistant for up to a month under typical cell culture conditions, and that the synthesis method is compatible with a range of patterning techniques from the nano- to the micro-scale, which enables the patterning of cells in a biologically relevant milieu over extended periods of time.

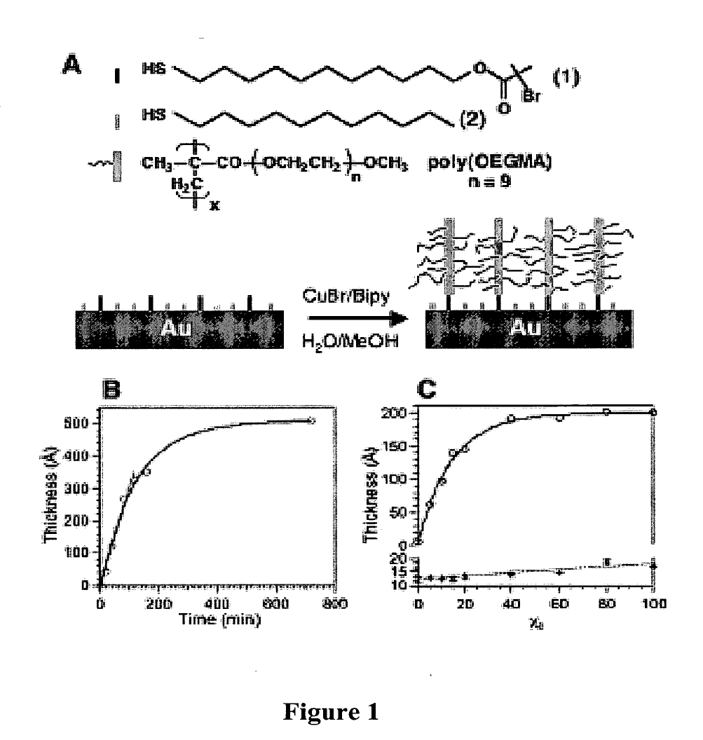

Surface-initiated polymerization of an (EG)ₙ-functionalized polymer brush was carried out from an alkanethiol SAM on gold, as follows (FIG. 1A): ω-mercaptoundecyl bromoisobutyrate (1) was synthesized as previously described (Jones, D. M., Brown, A. A., Huck, W. T. S., Langmuir 2002, 18, 1265) and a SAM of (1) was formed by immersion of a freshly prepared gold substrate in an ethanol solution of (1) (Nuzzo, R. G., Allara, D. L., J. Am. Chem. Soc. 1990, 105, 4481); in some experiments mixed SAMs were also prepared, where (1) was diluted with 1-undecanethiol (2) to vary the polymer brush density. SAMs of (1) present a terminal bromoisobutyrate moiety, which was utilized as a covalently tethered initiator for surface-initiated atom transfer radical polymerization (SI-ATRP) on gold. We chose SI-ATRP to grow polymer brushes from the surface, because ATRP is a living polymerization (Matyjaszewski, K. & Xia, J. H., Chem. Rev. 2001, 101, 2921), which provides control over the chain length and surface density of the polymer graft, and can also be used for the surface-initiated synthesis of block copolymers (Matyjaszewski, K. et al., Macromolecules 1999, 32, 8716), in relatively benign solvents under ambient conditions.

The polymerization was carried out in an oxygen-free environment, using CuBr/bipyridine as catalyst in a water/methanol mixture with oligoethylene glycol methyl methacrylate (OEGMA) (3) as the monomer (FIG. 1A). “Bottle” brushes of poly(OEGMA) were synthesized from a pure SAM of (1) on gold as a function of reaction time, and the thickness of the brushes were measured by ellipsometry (FIG. 1B). A linear fit of thickness against reaction time was found for a reaction time of up to 120 min (the dashed line in FIG. 1B, R²=0.98). A deviation from linear fit to exponential fit (the continuous curve in FIG. 1B) was observed for longer reaction time, and could be due to slow leakage of oxygen into the reaction system and/or increased steric interference to chain growth for longer polymer brushes.

The sessile water contact angle of the polymer surface was 42.3±0.6° which is significantly different from the water contact angle of 74.0±0.4° measured for the SAM of (1). The composition of these brushes was determined by XPS. An atomic O/C ratio of 0.33 was measured by XPS for a poly(OEGMA) brush, grown from a pure SAM of (1) with an ellipsometric thickness of 15.2 nm, and the high-resolution C1s spectrum of the same brush yielded a CHx/C—O—R/COOR ratio of 3/12.8/1.3. Both the level of oxygen incorporation and relative concentration of ether species is somewhat lower than the theoretical value of 0.48 for the atomic O/C ratio and the CHₓ/C—O—R/COOR ratio of 3/19/1 for pure poly(OEGMA); the lower amount of C—O—R species than expected from the stoichiometry could, in part, be due to the fact that the contribution of the SAM was not included in the calculation of the carbon moieties and the presence of contaminants.

The thickness of the mixed SAMs showed a linear increase with an increase in χ₁. We also varied the brush density on the surface by systematically varying the initiator coverage on the surface by preparing mixed SAMs of (1) and (2). SI-ATRP was carried out on these mixed SAMs for 40 min at room temperature. The thickness of the binary SAMs increased linearly with the increase in the mole fraction of (1) (χ₁) in solution (the dashed line in FIG. 1C, R²=0.89). Ellipsometry showed that the thickness of the polymer brushes reached a steady state of ˜20 nm at χ₁=0.6; beyond this value no further increase of film thickness was observed for the same SIP time (the continuous curve in FIG. 1C).

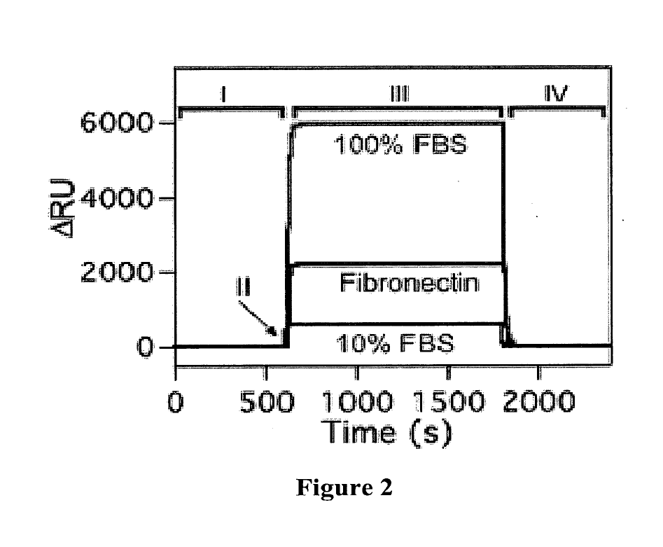

Motivated by the observation that (EG)ₙ-SH SAMs resist protein adsorption and cell adhesion, we examined the adsorption of different proteins onto ˜15 nm thick poly(OEGMA) brushes synthesized from a pure SAM of (1) on gold by SPR. We observed no protein adsorption onto the poly(OEGMA) brushes either from pure solutions of fibronectin (1 mg ml⁻¹), 10% fetal bovine serum (FBS, commonly used in cell culture), or 100% FBS (FIG. 2). The SPR response in ΔRU units was −3.2±2.9 (fibronectin), −4.6±4.6 (10% FBS) and −0.4±2.8 (100% FBS; n=3 for each protein), respectively. The small negative values of the SPR signal are simply a consequence of the fact that the SPR response is normalized to zero initially, so that the negative values of the SPR signal at the conclusion of the protein adsorption experiment are due to the −3×10⁻³ ΔRU s⁻¹ baseline drift of the instrument, which translates to a SPR signal of −5 ΔRU over the course of each experiment. These results indicate that the SPR signal from the surface of the poly(OEGMA) brushes after exposure to protein followed by a buffer wash is at or below the 0.1-1 ng cm⁻² detection limit of the Biacore X SPR instrument (BIAtechnology Handbook (Pharmacia Biosensor AB, Sweden), 1994).

These results are notable because they demonstrate that poly(OEGMA) brushes are exceptionally resistant to the adsorption of “sticky” proteins such as fibronectin and of proteins from a complex and concentrated protein mixture such as FBS. Theoretical and experimental studies by Grunze and colleagues on the origin of the non-fouling properties of (EG)ₙ-SH SAMs on gold have indicated that their protein resistance is controlled by two primary structural features: terminal hydrophilicity of the head-group combined with the formation of a dense, but disordered (EG)ₙ brush with significant penetration of water into the (EG)ₙ-SH SAMs (Wang, R. L. C., Kreuzer, H. T., Grunze, M., J. Phys. Chem. B 1997, 101, 9767; Pertsin, A. J., Hayashi, T., Grunze, M., J. Phys. Chem. B 2002, 106, 12274; Schwendel, D. et al., Langmuir 2003, 19, 2284. d) Herrwerth, S., Eck, W., Reinhardt, S., Grunze, M., J. Am. Chem. Soc. 2003, 125, 9359). These features, we believe, are also likely to be recapitulated by these polymer brushes and may explain their protein resistance, though experimental confirmation will require detailed characterization of the interfacial structure of the polymer brushes in the hydrated state.

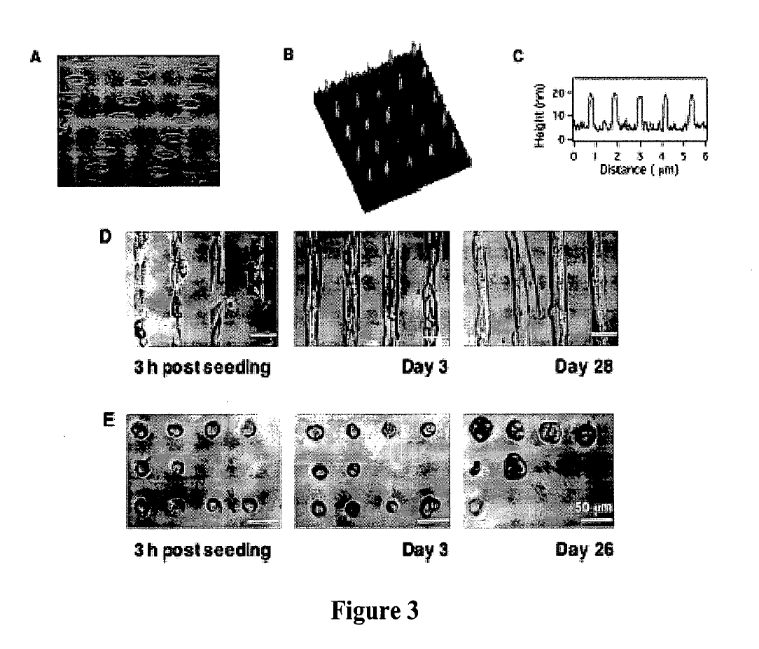

Because the poly(OEGMA) “bottle” brushes are grown from an initiator terminated alkanethiol SAM on gold, this “grafting from” or “in situ” synthesis is also compatible with methods used to pattern SAMs on gold, as shown previously by several groups (see, e.g., Zhao, B., Brittain, W. J., Prog. Polym. Sci. 2000, 25, 677, and references therein; Shah, R. R., et al, Macromolecules 2000, 33, 596; Jones, D. M., Huck, W. T. S., Adv. Mater. 2001, 13, 1256; Hyun, J., Chilkoti, A., Macromolecules 2001, 34, 5644; Tomlinson, M. R., Wu, T., Efimenko, K., Genzer, J. Polymer Preprints 2003, 44, 468; Schmelmer, U. et al. Angew. Chem. Int. Ed. 2003, 42, 559). We fabricated patterns of poly(OEGMA) at the micron scale by microcontact printing (uCP) (Kumar, A., Whitesides, G. M., Appl. Phys. Lett. 1993, 63, 2002) and at the nanometer scale by dip-pen nanolithography (DPN) (Piner, R. D., Zhu, J., Xu, F., Hong, S., Mirkin, C. A. Science 1999, 283, 661; Hyun, J., Ahn, S. J., Lee, W. K., Chilkoti, A., Zauscher, S., Nano Lett. 2002, 2, 1203). In brief, thiol (1) was patterned on a gold surface either by a PDMS stamp (μCP) or by an atomic force microscopy (AFM) tip (DPN) that was inked with (1). The unpatterned regions of bare gold were in some instances backfilled by incubation with (2) to form a hydrophobic CH3-terminated SAM or were deliberately left bare. SI-ATRP of OEGMA was then carried out on the patterned surface. The poly(OEGMA) patterns were characterized by scanning electron microscopy (SEM) and AFM. A representative SEM image of a microstructured surface in which the background was patterned by μCP of (1) followed by SI-ATRP of OEGMA (160 min polymerization time) is shown in FIG. 3A.

These polymer structures, grown in situ, can also be reduced to the nanoscale, as shown by the AFM image in FIG. 3B of a periodic array of poly(OEGMA) spikes grown from the surface by SI-ATRP of OEGMA (160 min polymerization time) following DPN of (1) onto gold. FIG. 3C, a line profile across the surface, shows that these polymeric nanostructures are ˜90 nm in diameter and ˜14 nm in height. We also note, parenthetically, that these results are the first demonstration, to our knowledge, that polymeric nanostructures can be grown, in situ from a surface by combining DPN with SIP. A different approach was also recently reported for the in situ fabrication of polystyrene nanostructures on a surface, initiated from a free-radical initiator that was patterned by nanoscale stencil masks (Schmelmer, U. et al. Angew. Chem. Int. Ed. 2003, 42, 559).

Micropatterns in which the background was patterned with a poly(OEGMA) brush (FIG. 3A), and the features were backfilled with a SAM of (2) were then incubated with fibronectin, a cell-adhesive protein (Horbett, T. A., Colloid Surface B 1994, 2, 225.). The lack of adsorption of fibronectin onto the poly(OEGMA) background, and its avid adsorption onto the SAM of (2) forms the basis of patterning cells, directed by the spatial localization of fibronectin. The patterned surfaces were then incubated with NIH 3T3 fibroblasts in 10% FBS for 3 h, washed to remove non-adherent cells, and then periodically observed under a light microscope. We observed good retention of cellular patterns for up to 30 days (FIGS. 3D and 3E) especially for patterns of isolated cells on circles. For cells that were patterned in stripes, we observed that some adjacent stripes of patterned cells merged after −0.10 days in culture. In contrast, cellular patterns, on (EG)ₙ-SH SAMs on gold, have been reported to degrade after ˜1 week in. culture (Mrksich, M., Dike, L. E., Tien, J., Ingber, D. E., Whitesides, G. M., Exp. Cell Res. 1997, 235, 305).

To our knowledge, these results are the first demonstration of the synthesis of a “nonfouling” polymer brush by surface-initiated polymerization of a macromonomer, and show that polymer brushes of tunable thickness in the range of 5-50 nm can be easily prepared by this method. The system described here recapitulates in a polymer brush some of the key features of (EG)ₙ-SH SAMs, namely the high density of oligoethylene glycol moieties (although, the architecture, we note is considerably different), the ease of fabrication stemming from chemical self-assembly on gold, easy characterization of the polymer brushes via optical evanescent techniques, and its compatibility with “soft” lithography and dip-pen nanolithography. The fabrication strategy reported here is complementary to previous approaches to create nonfouling surfaces by physical deposition of amphiphilic copolymers of methyl methacrylate (MMA) and OEGMA onto different substrates (Irvine, D. J., Griffith, L. G., Mayes, A. M., Biomacromolecules 2001, 2, 85; Jiang, X., Hammond, P. T., Polym. Mater. Sci. Eng. 2001, 84, 172; Hyun, J., et al., Langmuir 2002, 18, 2975), as well as the fabrication of nonfouling microstructures by μCP of the amphiphilic poly(MMA/OEGMA) copolymer (Hyun, J., Ma, H., Zhang, Z., Beebe Jr, T. P., Chilkoti, A., Adv. Mater. 2003, 15, 576). Together, the physical printing of microstructures of a nonfouling amphiphilic copolymer of MMA and OEGMA by μCP reported previously (Id.), and SIP of the OEGMA homopolymer from micropatterned, tethered initiators reported here provide an ensemble of techniques which allow the fabrication of nonfouling, polymeric micro- and nano-structures whose topography can be systematically controlled from several naometers (via SIP) to several microns (via physical printing). We believe that these “nonfouling” surfaces and topographical structures have utility in the design of experimentally useful model systems to investigate the response of cells to chemical and topographical cues, in addition to a wide range of applications in bioanalytical devices.

### Methods:

Synthesis of ω-mercaptoundecyl bromoisobutyrate (1).

The initiator (1) was synthesized using a previously published procedure with some modifications (Jones, D. M., Brown, A. A., Huck, W. T. S., Langmuir 2002, 18, 1265), Mercaptoundecanol (0.9590 g, 4.69 mmol), pyridine (0.35 ml, 4.27 mmol) and dry dichloromethane (30 ml) were added to a 100 ml round flask with a stir bar. The mixture was cooled down to 0° C., followed by dropwise addition of ice-cold bromoisobutyryl bromide (0.53 ml, 4.27 mmol, in 1 ml CH₂Cl₂ with 10 mg dimethylaminopyridine (DMAP)). After stirring at 0° C. for 1 h, the reaction was continued for another 16 h at room temperature. Water (30 ml) and toluene (15 ml) were added to the mixture for extraction. The aqueous phase was further extracted with toluene (2×30 ml). The organic phase was concentrated by rotoevaporation to remove toluene. The resulting crude extract was dissolved in ether (40 ml) and washed with a saturated ammonium chloride solution (3×40 ml), and dried over MgSO₄. Removal of the ether resulted in a yellowish oil, which was passed through a column (silica gel, neutral, hexane with 2% triethylamine as eluent) and then vacuum dried overnight. The final product was a colorless oil (1), obtained in high purity and with high yield (1.4040 g, 93.1% yield). ¹H NMR (300 MHz, CDCl₃): 4.15 (t, J=6.6, 2H, OCH₂), 2.50 (q, J=7.5, 2H, SCH₂), 1.92 (s, 6H, CH₃), 1.57-1.68 (m, 4H, CH₂), 1.26-1.36 (m, 16H, CH₂). ¹³C NMR (300 MHz, CDCl₃): 171.7 (C═O), 66.1 (OCH₂), 56.0 (C), 34.0 (SCH₂), 30.8 (CH₃), 29.4 (CH₂), 29.1 (CH₂), 29.0 (CH₂), 28.3 (CH₂), 25.7 (CH₂), 24.6 (CH₂).

Preparation and Patterning of SAMs.

SAMs of (1) were prepared by immersing goldcoated silicon chips (orientation (100), Umicore Semiconductor Processing, MA; 1.5×1.5 cm₂, primed with 50 Å Cr and then coated by thermal evaporation with 2000 Å Au for ellipsometry or 500 Å for SPR or cell culture) into a 1 mM solution of (1) in ethanol overnight. Mixed SAMs of (1) and (2) were prepared by immersing the chips into a 1 mM solution (total concentration) of the two thiols. Polydimethysiloxane (PDMS) stamps with different feature sizes were prepared as described previously (Irvine, D. J., Griffith, L. G., Mayes, A. M., Biomacromolecules 2001, 2, 85; Jiang, X., Hammond, P. T., Polym. Mater. Sci. Eng. 2001, 84, 172; Hyun, J., et al., Langmuir 2002, 18, 2975; Hyun, J., Ma, H., Zhang, Z., Beebe Jr, T. P., Chilkoti, A., Adv. Mater. 2003, 15, 576) and inked with (1). The stamps were brought into contact with a gold surface (1×1 cm²) to transfer the thiols to the surface. In some instances, an after micro-contact (uCP) gold surface was backfilled by incubation in a 1 mM solution of (2) for 5 min.

Dip-Pen Nanolithography.

Thiol (1) was patterned on a gold surface with dip-pen nanolithography (DPN), using an atomic force microscope (AFM) (MultiMode™, Digital Instruments). First, an AFM cantilever (silicon nitride cantilever, 0.05 N m⁻¹, Digital Instruments) was incubated in a solution of (1) in degassed acetonitrile for 1 min. The relative humidity during patterning ranged from 35% to 55%. Patterns of (1) were generated with writing speeds up to 8 um^{s-1} and nanoarrays of periodic features ranging from 100 to 2000 nm were routinely patterned by programming the XY motion of the AFM tube scanner through a customized nanolithography program (NanoScript™, Digital Instruments). Accurate patterned areas were repeatedly located by pixel correlation using still-video micrographs captured during lithography. The feature height after SI-ATRP of comb polymer was determined from line profiles of AFM height images.

Surface Initiated Atom Transfer Radical Polymerization.

Gold-coated Si chips, modified with a SAM of (1) or mixed SAMs of (1) and (2), were thoroughly rinsed with methanol to remove physisorbed initiator (1), and placed in a 100 ml flask that was connected to a′50 ml dropping funnel (with pressure-equalization arm). The system was evacuated for 30 min and purged with nitrogen thrice. Next, CuBr (143 mg, 1.0 mmol), bipyridine (312 mg, 2.0 mmol), and a mixture of deinoized water (degassed, 3 ml) and methanol (12 ml) were added to a 50 ml round-bottom flask with a stir bar. The mixture was stirred and the macromonomer OEGMA (8 g, 16.7 mmol) was added and the dark red solution was bubbled with nitrogen for 30 min. The mixture was transferred by a syringe to the funnel and purged with nitrogen for 5 min. Polymerization was initiated by adding the mixture into the flask and was continued for a specified time (10 to 720 min) under nitrogen purge. The samples were pulled out of the solution to stop the polymerization, rinsed with methanol and dried under flowing nitrogen.

Ellipsometry.

Film thickness was measured on a M-88 spectroscopic ellipsometer (J. A. Woollam Co., Inc) at angles of 65°, 70° and 75° and wavelengths from 400 nm to 800 nm. A Cauchy layer model provided with the instrument was used for all organic films, and the ellipsometric data were fitted for thickness of SAMs and poly(OEGMA) film with fixed (An, Bn) values of (1.45, 0), and (1.46, 0), respectively (Prime, K. L., Whitesides, G. M., J. Am. Chem. Soc. 1993, 15, 10714).

Contact Angle Measurement.

The sessile water contact angle measurements were performed on a Rome-Hart goniometer (100-00, Mountain Lakes, N.J.) using deionized water. Substrates were rinsed with methanol and deionized water and dried under a stream of nitrogen before measurement. The contact angle (and ellipsometric thickness) for each sample was independently measured at three different locations and is reported as the average±sd.

X-ray Photoelectron Spectroscopy.

XPS studies were performed on a VG ESCALAB 200i-XL electron spectrometer (VG Scientific Ltd., U.K.). Monochromatic Al K_{α} X-rays (1486.7 eV) were employed. Operation conditions for the X-ray source were 400 um nominal X-ray spot size (FWHM) operating at 15 kV, 8.9 mA for both survey and high-resolution spectra. Survey spectra, from 0 to 1200 eV binding energy, were recorded at 100 eV pass energy with an energy step of 1.0 eV, a dwell time of 100 ms, for one scan. High-resolution spectra were recorded at 20 eV pass energy with an energy step of 0.1 eV, a dwell time of 1.2 s, with a typical average of 12 scans. The operating pressure of the spectrometer was typically ˜10⁻⁹ mbar. All data were collected and analyzed using the Eclipse™ data system software. The electron flood gun was not used in these measurements.

Scanning Electron Microscopy.

A Philips XL 30 ESEM TMP was operated at 30.0 kV in conventional SEM mode to image the micropatterned polymer brushes on gold.

Surface Plasmon Resonance.

Protein adsorption was measured by surface plasmon resonance (SPR) spectroscopy on a Biacore X instrument (Biacore AB, Sweden). Blank SPR chips were prepared as previously described (Nath, N., Chilkoti, A., J. Am. Chem. Soc. 2001, 123, 8197). In brief, glass coverslips were primed with 30 Å Cr and then coated with 500 Å Au. After coating, they were cut into small pieces (0.8×1.0 cm²) and immersed into 1 mM solution of (1) overnight. Chips were coated with a poly(OEGMA) layer by 40 min of SI-ATRP and were immersed in MeOH for 2 h. They were then glued to empty Biacore cassettes using water-insoluble double-side sticky tape (3M Inc.) and docked into the instrument. After priming with PBS buffer (pH=7.4, Gibco™), protein solutions were flowed over the polymer surface at a flow rate of 2 ul min^{d} for 20 min at 25° C., followed by washing with PBS to remove loosely adsorbed protein.

Cell Patterning.

NIH 3T3 fibroblasts were grown in DMEM with 10% calf serum (Gibco BRL) supplemented with 100 units ml-1 penicillin, 100 ug ml⁻¹ streptomycin, and 7.5 mM HEPES at 37° C. in 5% CO₂. Cells near confluence were detached from the tissue culture flask using 0.05% trypsin-EDTA (Gibco BRL) and seeded onto micropatterned samples or controls (bare gold or full coverage of poly(OEGMA) by SIATRP) at a density of 30,000 cells cm⁻². The cell culture medium was changed 3 h postseeding to remove floating, dead cells, and every 3 days thereafter, and the cells were imaged at that time under reflective light microscopy (Vertical Fluorescence Model 2071, Warner-Lambert Tech. Inc., Buffalo, N.Y.).

The foregoing is illustrative of the present invention, and is not to be construed as limiting thereof. The invention is defined by the following claims, with equivalents of the claims to be included therein.

1.-99. (canceled)

100. A method of preparing a nonfouling polymer brush, comprising surface-initiated polymerizing a macromonomer to form the nonfouling polymer brush.

101. The method of claim 100, wherein the nonfouling polymer brush has a thickness of from about 15-100 nm.

102. The method of claim 101, wherein the nonfouling polymer brush has a length of from 5 to 100 nm.

103. The method of claim 102, wherein the macromonomer comprises a monomeric core group having a protein-resistant head group coupled thereto.

104. The method of claim 103, wherein the monomeric core group comprises a vinyl group.

105. The method of claim 103, wherein the protein-resistant head group comprises an oligoethylene glycol.

106. The method of claim 103, wherein the protein-resistant head group comprises a polyethylene glycol.

107. The method of claim 103, wherein the protein-resistant head group comprises a kosmotrope.

108. The method of claim 103, wherein the protein-resistant head group comprises from 3 to 20 ethylene glycol units.

109. The method of claim 103, wherein the protein-resistant head group comprises—(ethylene glycol)₆OH.

110. The method of claim 103, wherein the macromonomer comprises oligoethylene glycol methyl methacrylate.

111. The method of claim 100, further comprising conducting the surface-initiated polymerizing in the presence of a monomer that is not a macromonomer.

112. The method of claim 111, wherein the monomer is methylmethacrylate.

113. The method of claim 111, wherein the monomer comprises a vinyl group.

114. The method of claim 100, comprising forming a plurality of nonfouling polymer brushes.

115. The method of claim 101, comprising forming a plurality of nonfouling polymer brushes.

116. The method of claim 102, comprising forming a plurality of nonfouling polymer brushes.

117. The method of claim 103, comprising forming a plurality of nonfouling polymer brushes.

118. The method of claim 107, comprising forming a plurality of nonfouling polymer brushes.

119. The method of claim 108, comprising forming a plurality of nonfouling polymer brushes.

120. The method of claim 111, comprising forming a plurality of non-fouling polymer brushes.

121. The method of any one of claims 100-111, wherein the polymer brush is non-fouling if a plurality of polymer brushes i) after being primed with phosphate buffered saline (PBS) solution having a pH of 7.4 for 10 minutes; ii) then being exposed to a fibronectin solution at a concentration of 1 mg fibronectin per ml for 20 minutes at 25° C.; and iii) then being rinsed for 10 minutes with a PBS solution having a pH of 7.4 for 10 minutes; when evaluated by surface plasmon resonance (SPR), shows a fibronectin absorption of equal to or below 1 ng per cm².

122. The method any one of claims 100-111, wherein the polymer brush is non-fouling if a plurality of polymer brushes i) after being primed with phosphate buffered saline (PBS) solution having a pH of 7.4 for 10 minutes; ii) then being exposed to a 10% solution of fetal bovine serum (FBS) for 20 minutes at 25° C.; and iii) then being rinsed for 10 minutes with a PBS solution having a pH of 7.4 for 10 minutes; when evaluated by surface plasmon resonance (SPR), shows a fetal bovine serum absorption of equal to or below 1 ng per cm².

123. A nonfouling polymer brush made by the method of claim 121.

124. A nonfouling surface comprising nonfouling a polymer brush made by the method of claims 123.

125. A nonfouling polymer brush made by the method of claim 122.

126. A nonfouling surface comprising a nonfouling polymer brush made by the method of claim 125.

127. The method of claim 100, wherein the polymer brush is non-fouling if a plurality of polymer brushes i) after being primed with phosphate buffered saline (PBS) solution having a pH of 7.4 for 10 minutes; ii) then being exposed to a 100% solution of fetal bovine serum (FBS) for 20 minutes at 25° C.; and iii) then being rinsed for 10 minutes with a PBS solution having a pH of 7.4 for 10 minutes; when evaluated by surface plasmon resonance (SPR), shows a fetal bovine serum absorption of equal to or below 1 ng per cm².
